(19) United States
(12) Patent Application Publication
(10) Pub. No.: US 2021/0213073 A1
(43) Pub. Date: Jul. 15, 2021

(54) APPLICATION OF B. FRAGILIS OR AKKERMANSIA MUCINIPHILA IN PREPARATION OF DRUG FOR PREVENTING OR TREATING TUMOR

(71) Applicant: REVAISSANT (SHENZHEN) BIOSCIENCES CO., LTD., Shenzhen, Guangdong (CN)

(72) Inventor: Gucheng ZENG, Guangzhou City (CN)

(21) Appl. No.: 17/054,836

(22) PCT Filed: Jun. 1, 2018
(86) PCT No.: PCT/CN2018/089561

§ 371 Date: Nov. 12, 2020

(30) Foreign Application Priority Data
May 18, 2018 (CN) 201810479187.6

(51) Int. Cl. A61K 35/74; A23L 33/135

(52) U.S. Cl. CPC A61K 35/74; A23L 33/135

(57) ABSTRACT

Provided is a use of Bacteroides fragilis or Akkermansia muciniphila in the preparation of a drug for preventing or treating tumors, wherein the drug promotes infiltration or accumulation of CD8 positive cytotoxic T lymphocyte in tumor microenvironment. Also provided is a use of a pharmaceutical composition, foodstuff, health product or food additive in preventing and/or treating tumors, wherein the pharmaceutical composition, foodstuff, health product or food additive includes Bacteroides fragilis or Akkermansia muciniphila, and promotes infiltration or accumulation of CD8 positive cytotoxic T lymphocytes in tumor microenvironment.

### CROSS REFERENCE TO RELATED APPLICATIONS

This application claims priority to PCT Application No. PCT/CN2018/089561, having a filing date of Jun. 1, 2018, which is based on Chinese Application No. 201810479187.6, having a filing date of May 18, 2018, the entire contents both of which are hereby incorporated by reference.

### FIELD OF TECHNOLOGY

The following relates to the technical field of biomedicine, particularly, it relates to a use of Bacteroides fragilis or Akkermansia muciniphila in the preparation of a drug for preventing and/or treating tumors.

### BACKGROUND

Cancer has become the “first killer” of human beings. It is predicted by The Global Cancer Report 2014, published by the World Health Organization (WHO) that the global cancer cases will increase rapidly, and will increase year by year from 14 million people in 2012 to 19 million people in 2025, and to 24 million in 2035. Every year, there are about 7 million new cancer patients worldwide, and about 5 million tumor patients die, indicating that one person dies from tumor every 6 seconds. Chemotherapy is currently recognized as the main treatment method, and its main objective is to kill cancer cells in the body. However, chemotherapeutic drugs also damage normal human cells while killing cancer cells. Breast cancer is currently the most common type of malignant tumors, and it is also the most common disease that takes away the health and life quality of women. Methods of treating breast cancer are mainly surgical treatment and chemotherapy. With the development of personalized treatment through genetic diagnosis, breast cancer patients have good treatment results. However, due to various reasons, most patients have unsatisfactory curative effect. Therefore, the research of anti-breast cancer drugs is of great significance. In the Annual Report of the ASCO Cancer Research Progress in 2016 published by the American Society of Clinical Oncology (ASCO) on Feb. 4, 2016, immunotherapy was named as the biggest progress in cancer research in 2015. Just as Dr. Julie M. Vose, the chairman of ASCO, said, “Immunotherapy is the most revolutionary breakthrough in the cancer field, and this new therapy not only can improve the lives of patients, but also give the direction for future research”. Current tumor immunotherapy will become the fourth major cancer treatment after surgery, radiotherapy and chemotherapy. Therefore, it has become a worldwide research hotspot to develop safe, low-priced, highly effective and low side-effect cancer immune drugs.

Currently, there are two tumor immunotherapeutic technologies showing good clinical effects. The first one is an adoptive cellular immunotherapy, which plays a role in anti-cancer treatment by obtaining the immune cells in body of the patients, then inducing to cells with killing effects, for example, chimeric antigen receptors T cell (CAR-T), etc., based upon the properly of tumor target antigen, followed by transfusing the cells with killing effects back into the body. The second one is an antibody-targeted therapy, wherein targeted drugs inhibit cancer cells by interacting with a specific molecule target that is necessary during the growth or metastasis of tumors. Nowadays, there are antibody drugs for blocking T cell exhausted molecule such as CTLA4, PD-1, PD-L1, etc. Although these blocking antibody tumor treatments show clinical effects on some kinds of tumors for some patients, the blocking antibody drugs have low or no effects for a large proportion of patients, and a lack of the infiltration and accumulation of CD8 positive cytotoxic T lymphocytes (CD⁸⁺ T cells, for short) in tumor microenvironment is one of key reasons for the poor immunotherapeutic effect. How to promote the infiltration or accumulation of CD8⁺ T cells in tumor microenvironment becomes a key scientific and technical problem that needs to be solved urgently in tumor immunotherapy, considering that CD8⁺ T cells do not only have a key function of killing tumor cells directly but also improve efficacy of the immunotherapy by significantly enhancing the patient’ responses to immunotherapy techniques, for example CAR-T and antibody drugs for blocking T cell exhausted molecule.

Uses of bacteria in cancer treatment can be traced back to the late nineteenth century, and there are even earlier reports on efficacy of bacteria in the treatment for cancer. Probiotics are active microorganisms that are beneficial to the host, and after probiotics are ingested into the human or animal body, they can settle on intestinal mucosa, establish intestinal microbiota and prevent harmful microorganism from adhering thereto Probiotics also can keep people or animal healthy by maintaining natural intestinal microbiota and promoting the formation of healthy and viable microbial preparation for individual organisms. Currently, more and more researchers have focused on probiotics and gradually realized their powerful therapeutic effects. A lot of the latest works about bacteriotherapy for cancer focus on non-pathogenic strains. Bifidobacterium is a non-pathogenic and obligate anaerobic bacterium, and has been successfully used for targeting tumors and used as a therapeutic carrier, but without showing oncolysis. In recent years, some researches apply Escherichia coli and pneumobacillus for cancer/tumor of intestine and lung, respectively, as “site-specific immunomodulators” which play a more significant role in inhibiting tumor growth. However, uses of probiotics or intestinal bacteria for promoting the infiltration and accumulation of CD8⁺ T cell in tumor microenvironment haven't been reported.

Bacteroides fragilis is a Gram-negative, rod-shaped, non-motile, and non-spore-forming obligate anaerobic bacterium, having obtuse and hyperchromatic ends as well as a capsule. The Bacteroides fragilis can be classified into an enterotoxigenic type and a non-enterotoxigenic type. As a part of the normal intestinal flora of humans and animals. Bacteroides fragilis mainly exists in the colon, and besides, it can also colonize and grow in the respiratory tract, the gastrointestinal tract and the urogenital tract. Numerous researches have shown that Bacteroides fragilis has a good effect on the prevention and treatment of acute and chronic enteritis, dysbacteriosis, upper respiratory infection and neurosis, etc.

Akkermansia muciniphila (phylum Verrucomicrobia) is an anaerobic, atrichous, non-spore-forming, non-motile, Gram-negative, and oval-shaped gut bacterium, with a certain anaerobic ability. Akkermansia muciniphila, accounting for 1-3% of the total amount of gut microorganism, is one of the dominant intestinal flora of human, and the Akkermansia muciniphila colonized in mucous layer can specifically degrade mucoprotein. Current studies have found that Akkermansia muciniphila colonization abundance in humans is negatively correlated with obesity and type 2 diabetes, and Akkermansia muciniphila colonization is important for metabolisms in organisms.

However, uses of intestinal bacterium comprising Bacteroides fragilis and/or Akkermansia muciniphila for promoting the infiltration and/or accumulation of CD8⁺ T cells in tumor microenvironment to prevent and/or treat tumors haven't been reported.

### SUMMARY

In view of the difficulty of lacking the infiltration and/or accumulation of CD8 positive cytotoxic T lymphocytes (CD8⁺ T cells, for short) in tumor microenvironment for current tumor immunotherapy, it is to be provided a drug which can promote the infiltration or accumulation of CD8⁺ T cells in tumor microenvironment.

An aspect relates to a use of Bacteroides fragilis (B. fragilis) or Akkermansia muciniphila in the preparation of a drug for preventing and/or treating tumors, wherein the drug promotes the infiltration or accumulation of CD8⁺ T cells in tumor microenvironment.

The Bacteroides fragilis or Akkermansia muciniphila is any one selected from live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and/or culture supernatant of Bacteroides fragilis or Akkermansia muciniphila.

The tumors described in the present disclosure may be various solid tumors, for example, but are not limited to, breast cancer, or any one or more of liver, lung, skin, oral, esophagus, stomach, intestine, kidney, prostate, brain, nervous system, bladder, lymph, and pancreas tumors, such as solid tumors, for example lung cancer, melanoma tumor, liver cancer, prostate cancer, fibrosarcoma, bladder sarcoma, and glioma, etc.

It is another aspect to provide a method of promoting the infiltration and/or accumulation of CD8 positive cytotoxic T lymphocytes in tumor microenvironment to prevent and/or treat tumors.

The Bacteroides fragilis or Akkermansia muciniphila is any one selected from live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and/or culture supernatant of Bacteroides fragilis or Akkermansia muciniphila.

In some examples, the method of promoting the infiltration and/or accumulation of CD8⁺ T cells in tumor microenvironment to prevent and/or treat tumors is applied in combination with other methods of promoting the infiltration and/or accumulation of CD8⁺ T cells in tumor microenvironment. In some examples, the other methods of promoting the infiltration and/or accumulation of CD8⁺ T cells in tumor microenvironment to prevent and/or treat tumors include but are not limited to, chemotherapy, reflexotherapy, gene therapy, surgery, or a combination thereof.

It is another aspect to provide a therapeutic and prophylactic composition comprising Bacteroides fragilis or Akkermansia muciniphila. In some examples, the therapeutic and prophylactic composition includes Bacteroides fragilis or Akkermansia muciniphila. In some examples, the therapeutic and prophylactic composition does not include other microbial strains. In one aspect, the Bacteroides fragilis or Akkermansia muciniphila can inhibit tumor growth. In another aspect, the tumor is a solid tumor. In some examples, the tumor includes but is not limited to breast cancer.

According to one aspect of the present disclosure, it is another aspect to provide a pharmaceutical composition for preventing and/or treating tumors, wherein the pharmaceutical composition comprises a pharmaceutically effective amount of Bacteroides fragilis or Akkermansia muciniphila and a pharmaceutically acceptable carrier thereof, and can promote the infiltration and/or accumulation of CD8⁺ T cells in tumor microenvironment. The Bacteroides fragilis or Akkermansia muciniphila is an active ingredient.

In above pharmaceutical composition, the Bacteroides fragilis or Akkermansia muciniphila is any one selected from live bacterium of Bacteroides fragilis or Akkermansia muciniphila; genetically recombined, altered or modified, attenuated, chemically treated, physically treated or inactivated Bacteroides fragilis or Akkermansia muciniphila; lysate of Bacteroides fragilis or Akkermansia muciniphila; and/or culture supernatant of Bacteroides fragilis or Akkermansia muciniphila.

In the above pharmaceutical composition, the pharmaceutical composition can be made in any one or more of pharmaceutically acceptable dosage forms, including but are not limited to, tablet, capsule, oral liquid or freeze-dried powders.

In the above pharmaceutical composition, the pharmaceutical acceptable carrier is one of skim milk, lactose, glucose, sucrose, sorbitol, mannose, trehalose, starch, arabic gum, calcium phosphate, alginate, gelatin, calcium silicate, fine crystalline cellulose, polyvinylpyrrolidone, cellulose, water, syrup, methyl cellulose, methyl hydroxybenzoate, propyl hydroxybenzoate, talc, magnesium stearate, or mineral oil, or a mixture thereof.

It is another aspect to provide a foodstuff for treating and/or preventing tumors, wherein the foodstuff includes Bacteroides fragilis or Akkermansia muciniphila, and can promote the infiltration and/or accumulation of CD8 positive cytotoxic T lymphocytes in tumor microenvironment.

In the above foodstuff, the Bacteroides fragilis or Akkermansia muciniphila is any one selected from live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and/or culture supernatant of Bacteroides fragilis or Akkermansia muciniphila.

It is another aspect to provide a food additive for treating and/or preventing tumors, wherein the food additive includes Bacteroides fragilis or Akkermansia muciniphila, and can promote the infiltration and/or accumulation of CD8 positive cytotoxic T lymphocytes in tumor microenvironment.

In the above food addictive, the Bacteroides fragilis or Akkermansia muciniphila is any one selected from live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and/or culture supernatant of Bacteroides fragilis or Akkermansia muciniphila.

It is another aspect to provide a health product for treating and/or preventing tumors, wherein the health product includes Bacteroides fragilis or Akkermansia muciniphila, and can promote the infiltration and/or accumulation of CD8 positive cytotoxic T lymphocytes in tumor microenvironment.

In the above health product, the Bacteroides fragilis or Akkermansia muciniphila is any one selected from live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and/or culture supernatant of Bacteroides fragilis or Akkermansia muciniphila.

In the disclosure, a transplanted tumor research method is applied to create a mouse breast cancer model, through which the effects of Bacteroides fragilis or Akkermansia muciniphila are detected and identified. Through experiments, the disclosure proves that the Bacteroides fragilis or Akkermansia muciniphila can significantly inhibit breast cancer from surviving in vitro and can effectively inhibit the growth of transplanted tumors in mice, indicating that it is important to develop and apply Bacteroides fragilis or Akkermansia muciniphila in clinical treatment of tumors.

### BRIEF DESCRIPTION

Some of examples will be described in detail, with reference to the following figures, wherein like designations denote like members, wherein,

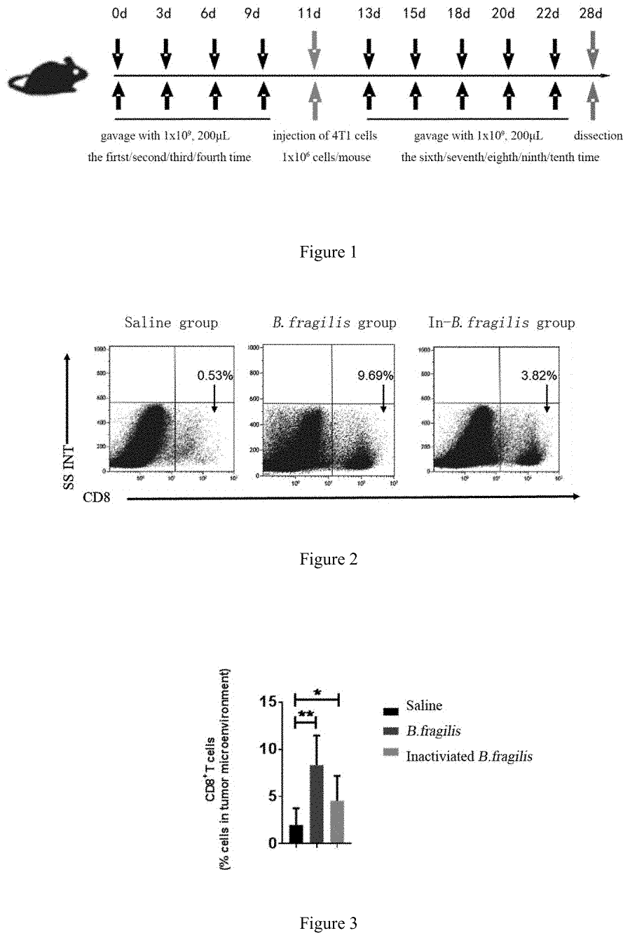

FIG. 1 is a schematic flow diagram of an experiment of detecting the effect of Bacteroides fragilis and inactivated Bacteroides fragilis in promoting the accumulation of CD8⁺ T cells in tumor microenvironment and in treatment in a mouse breast cancer model;

FIG. 2 is a typical analysis graph of flow cytometry of one mouse in each group after Bacteroides fragilis and inactivated Bacteroides fragilis are administrated to mice implanted with breast cancer cells, wherein right quadrant indicates CD8⁺ T cells, and figures of the right quadrant show the percentage of CD8⁺ T cells in total cells in tumor microenvironment;

FIG. 3 is a statistical analysis graph of the percentage of CD8⁺ T cells in total cells in tumor microenvironment after Bacteroides fragilis and inactivated Bacteroides fragilis are administrated to mice implanted with breast cancer cells;

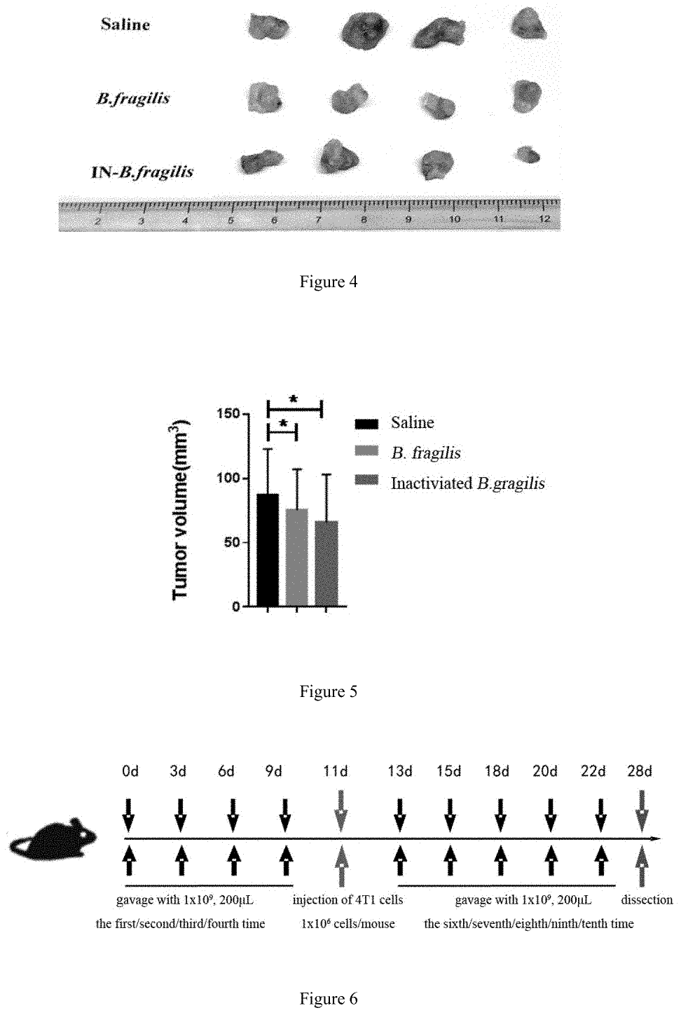

FIG. 4 is a comparison diagram of tumor size of breast cancer of mice after being treated with Bacteroides fragilis and inactivated Bacteroides fragilis;

FIG. 5 is a statistical analysis graph of the comparison diagram of tumor size of breast cancer in mice after being treated with Bacteroides fragilis and inactivated Bacteroides fragilis;

FIG. 6 is a schematic flow diagram of an experiment of detecting the effect of Akkermansia muciniphila in promoting the accumulation of CD8⁺ T cells in tumor microenvironment and in treatment in a mice breast cancer model;

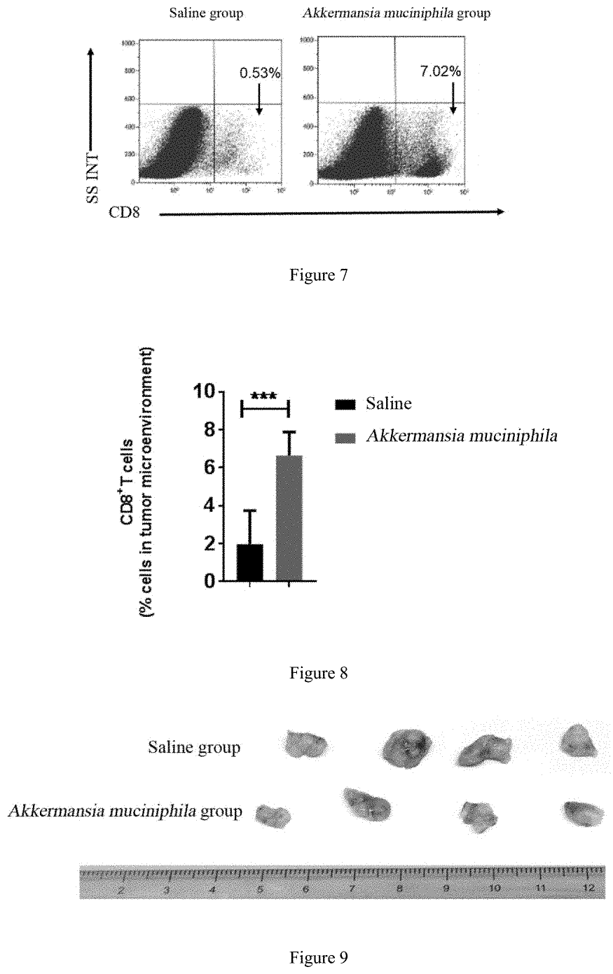

FIG. 7 is a typical analysis graph of flow cytometry of one mouse in each group after Akkermansia muciniphila is administrated to mice implanted with breast cancer cells, wherein right quadrant indicates CD8⁺ T cells, and figures of the right quadrant show the percentage of CD8⁺ T cells in total cells in tumor microenvironment;

FIG. 8 is a statistical analysis graph of the percentage of CD8⁺ T cells in total cells in tumor microenvironment after Akkermansia muciniphila is administrated to mice implanted with breast cancer cells;

FIG. 9 is a comparison diagram of tumor size of breast cancer of mice after being treated with Akkermansia muciniphila; and

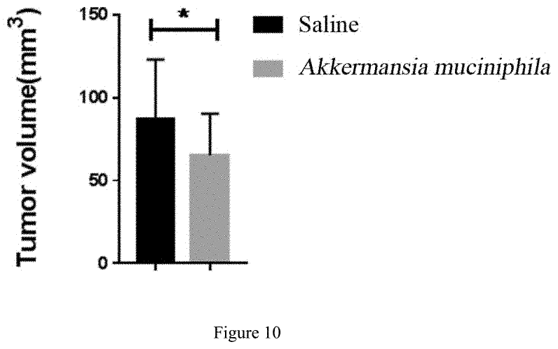

FIG. 10 is a statistical analysis graph of the comparison diagram of tumor size of breast cancer of mice after being treated with Akkermansia muciniphila.

### DETAILED DESCRIPTION

The present disclosure will be further described below with reference to the accompanied figures and examples. It should be pointed out that the Bacteroides fragilis or Akkermansia muciniphila for treating and/or preventing tumors in the present disclosure, or a pharmaceutical composition, foodstuff, health product or food additive containing the Bacteroides fragilis or Akkermansia muciniphila of the present disclosure is applied to the indications described above and exhibits the functions describe above, after being administrated to the subject. All dosage forms within the scope of the present disclosure have been tested, and their small parts are described hereinafter in the examples only for illustration, however, which should not be understood as a limitation of the present disclosure.

Bacteroides fragilis or Akkermansia muciniphila referred in the present disclosure includes but is not limited to any one selected from live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and/or culture supernatant of Bacteroides fragilis or Akkermansia muciniphila.

The tumor is a solid tumor. In some examples, the tumor includes but is not limited to breast cancer.

The present disclosure further provides an anti-tumor pharmaceutical composition containing a pharmaceutically effective amount of Bacteroides fragilis or Akkermansia muciniphila, wherein the so-called “pharmaceutically effective amount” is 10⁶-10¹⁰ CFU, for example 10⁹ CFU. The Bacteroides fragilis or Akkermansia muciniphila is any one selected from live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and/or culture supernatant of Bacteroides fragilis or Akkermansia muciniphila. The pharmaceutical composition includes but is not limited to tablet, capsule, oral liquid, or frizzed-dried powders. The pharmaceutically acceptable carrier includes but is not limited to one or more of skim milk, lactose, glucose, sucrose, sorbitol, mannose, trehalose, starch, arabic gum, calcium phosphate, alginate, gelatin, calcium silicate, fine crystalline cellulose, polyvinylpyrrolidone, cellulose, water, syrup, methyl cellulose, methyl hydroxybenzoate, propyl hydroxybenzoate, talc, magnesium stearate, or mineral oil.

The Bacteroides fragilis or Akkermansia muciniphila of the present disclosure can be made into foodstuffs, health products, or food additives, etc. The foodstuffs, health products or food additives contain any one selected from live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and/or culture supernatant of Bacteroides fragilis or Akkermansia muciniphila. The foodstuffs, health products or food additives are used for treating and/or preventing tumors.

### EXAMPLE 1

### Culture of Bacteroides Fragilis

Culture Method

Step 1: A cryopreserved Bacteroides fragilis strain (purchased from ATCC official website) was taken and then 200 uL of Tryptone Soya Broth (TSB) culture medium was added to redissolve it to obtain a bacterial solution. Subsequently, 20 uL of the bacterial solution was pipetted and streaked on a blood agar plate After an air exhaustion by an anaerobic jar gassing system, the agar plate was placed in an incubator and incubated anaerobically at 37° C. for 48 h;

Step 2: A monoclonal colony was selected to inoculate in 10 mL of TSB culture medium, and incubated anaerobically at 37° C. for 12 h;

Step 3: 1% (v/v) of strain was inoculated in 500 ml of TSB culture medium in a flask and incubated anaerobically at 37° C. for 48 h;

Step 4: After the bacterial solution was collected, it was centrifuged at 6000 rpm for 10 min, washed twice with saline. Finally, the bacterial sludge was redissolved with saline for later use and viable bacterium were counted.

### EXAMPLE 2

### Culture of Akkermansia Muciniphila

Culture Method

Step 1: A cryopreserved Akkermansia muciniphila strain (purchased from official website of ATCC) was taken and 200 uL of Tryptone Soya Broth (TSB) culture medium was added to redissolve it to obtain a bacterial solution. Subsequently, 20 uL of the bacterial solution was pipetted and streaked on a blood agar plate. After an air exhaustion by an anaerobic jar gassing system, the agar plate was placed in an incubator and incubated anaerobically at 37° C. for 48 h;

Step 2: A monoclonal colony was selected to inoculate in 10 mL of TSB culture medium, and incubated anaerobically at 37° C. for 12 h;

Step 3: 1% (v/v) of strain was inoculated in 500 ml of TSB culture medium in a flask and incubated anaerobically at 37° C. for 48 h;

Step 4: After the bacterial solution was collected, it was centrifuged at 6000 rpm for 10 min, washed twice with saline. Finally, the bacterial sludge was redissolved with saline for later use and viable bacterium were counted.

### EXAMPLE 3

An experiment of effect of Bacteroides fragilis in promoting the infiltration and/or accumulation of CD8⁺ cells in tumor microenvironment and in treating.

FIG. 1 is a schematic flow diagram of an experiment for detecting the effect of Bacteroides fragilis and inactivated Bacteroides fragilis in promoting the accumulation of CD8⁺ T cells in tumor microenvironment and in treatment.

1. Culture Method

A culture method of Bacteroides fragilis is the same as that in Example 1.

2. Sample Preparation

1) Preparation of a live strain of Bacteroides fragilis ZY-312

Step 1: A cryopreserved Bacteroides fragilis strain (purchased commercially) was taken and 200 uL of culture medium for cryopreserved strain was added to redissolve it to obtain a bacterial solution. Subsequently, 20 uL of the bacterial solution was pipetted and streaked on a blood agar plate. After an air exhaustion by an anaerobic jar gassing system, the agar plate was placed in an incubator and incubated anaerobically at 37° C. for 48 h;

Step 2: A monoclonal colony was selected to inoculate in 10 mL of TSB culture medium, and incubated anaerobically at 37° C. for 12 h.

Step 3: 1% (v/v) of strain was inoculated in 500 ml of TSB culture medium in a flask and incubated anaerobically at 37° C. for 48 h;

Step 4: After the bacterial solution was collected, it was centrifuged at 6000 rpm for 10 min, washed twice with saline. Finally, the bacterial sludge was redissolved with saline for later use and viable bacterium were counted.

2) Inactivated Bacteroides fragilis (In-B. fragilis)

The Bacteroides fragilis was heated in a water bath at 70° C. for 30 min to obtain inactivated bacteria solution of Bacteroides fragilis.

3) Lysate of Bacteroides fragilis

The bacterial solution of Bacteroides fragilis was cultured, and an ultrasonication was performed by an ultrasonic machine for 2 seconds every 5 seconds, the total ultrasonication lasts for 20 minutes, a lysate of Bacteroides fragilis was obtained.

4) Supernate of Bacteroides fragilis

The bacterial solution of Bacteroides fragilis was cultured and centrifuged by a centrifuge at 6000 rpm for 10 min. to obtain supernate of Bacteroides fragilis.

3. An experiment of prevention and treatment effect of Bacteroides fragilis on tumor in mice

Experimental animal: Thirty-six BALB/c mice aged 3-6 weeks in a good mental state were purchased from the Experimental Animal Center of Sun Yat-sen University. The mice were randomly separated into three groups, 12 mice in each group, the three groups were the control group (saline), the live bacteria gavage group (Bacteroides fragilis), and the inactivated bacteria gavage group (inactivated Bacteroides fragilis) respectively. The three groups of mice were administered with saline, Bacteroides fragilis, and inactivated Bacteroides fragilis by gavage with the density of 10⁹ CFU, respectively, and their body weight were measured daily. After 4TI mouse tumor (breast cancer) cells grew at logarithmic phase, the cells were digested with TE, and culture medium was neutralized. The cells were collected through centrifugation, and washed twice with DPBS to remove residual serum. After that, the cells were resuspended with DPBS. When the cell counting was completed, each mouse was subcutaneously inoculated with 10⁶ cells into right axilla and continually treated by gavage. Subsequently, tumor-bearing mice were killed. The tumor cells in situ were collected, and were detected by using flow cytometry to analyze the percentage of CD8+ T cells.

### EXAMPLE 4

An experiment of effect of Akkermansia muciniphila in promoting the infiltration and/or accumulation of CD8⁺ T cells in tumor microenvironment and treating tumors

FIG. 6 is a schematic flow diagram of an experiment of detecting the effect of Akkermansia muciniphila and inactivated Akkermansia muciniphila in promoting the accumulation of CD8⁺ T cells in tumor microenvironment and in treatment

1. Culture Method

A culture method of Akkermansia muciniphila is the same as that in Example 2.

2. Sample Preparation

1) Preparation of a live strain of Akkermansia muciniphila

Step 1: A cryopreserved Akkermansia muciniphila strain (purchased commercially) was taken and 200 uL of culture medium for the cryopreserved strain was added to redissolve it to obtain a bacterial solution. Subsequently, 20 uL of the bacterial solution was pipetted and streaked on a blood agar plate. After an air exhaustion by an anaerobic jar gassing system, the agar plate was placed in an incubator and incubated anaerobically at 37° C. for 48 h;

Step 2: A monoclonal colony was selected to inoculate in 10 mL of TSB culture medium, and incubated anaerobically at 37° C. for 12 h;

Step 3: 1% (v/v) of strain was inoculated in 500 ml of TSB culture medium in a flask and incubated anaerobically at 37° C. for 48 h;

Step 4: After the bacterial solution was collected, it was centrifuged at 6000 rpm for 10 min, washed twice with saline. Finally, the bacterial sludge was redissolved with saline for later use and viable bacterium were counted.

2) Inactivated Akkermansia muciniphila

The Akkermansia muciniphila was heated in a water bath at 70° C. for 30 min to obtain inactivated Akkermansia muciniphila.

3) Lysate of Akkermansia muciniphila

The bacterial solution of Akkermansia muciniphila was cultured, and an ultrasonication was performed by an ultrasonic machine for 2 seconds every 5 seconds, the total ultrasonication lasts for 20 minutes, a lysate of Akkermansia muciniphila was obtained.

4 ) Supernate of Akkermansia muciniphila

The bacterial solution of Akkermansia muciniphila was cultured and centrifuged by a centrifuge at 6000 rpm for 10 min, to obtain a supernate of Akkermansia muciniphila.

3. An experiment of prevention and treatment effect of Akkermansia muciniphila on tumor in mice

Experimental animal: Twenty-four BALB/c mice aged 3-4 weeks in a good mental state were purchased from the Experimental Animal Center of Sun Yat-sen University. The mice were randomly separated into two groups, 12 mice in each group, the two groups were the control group (saline) and the live bacteria gavage group (Akkermansia muciniphila), respectively. The two groups of mice were administered with saline and Akkermansia muciniphila by gavage with the density of 10⁹ CFU, respectively, and their body weight were measured daily. After 4TI mouse tumor (breast cancer) cells grew at logarithmic phase, the cells were digested with TE, and culture medium was neutralized The cells were collected through centrifugation, and washed twice with DPBS to remove residual scrum. After that, the cells were resuspended with DPBS. When the cell counting was completed, each mouse was subcutaneously inoculated with 10⁶ cells into right axilla and continually treated by gavage. Subsequently, tumor-bearing mice were killed The tumor cells in situ were collected, and were detected by using flow cytometry to analyze the percentage of CD8⁺ T cells.

Analysis of Test Results

FIG. 2 and FIG. 7 show typical flow cytometry test results of each mouse and FIG. 3 and FIG. 8 show statistical results of multiple mice in each group.

FIG. 2 is a typical analysis graph of flow cytometry of one mouse in each group after Bacteroides fragilis are administrated to mice implanted with breast cancer cells, wherein figures of the right quadrant show the percentages of CD8⁺ T cells in cells in tumor microenvironment. As shown from the quadrant graph in analysis graph of the flow cytometry, Bacteroides fragilis increases the relative amount of the CD8⁺0 T cells by about 20 times compared with the saline control group. FIG. 3 is a statistical analysis graph of the percentage of CD8⁺ T cells in cells in tumor microenvironment after Bacteroides fragilis are administrated to mice implanted with breast cancer cells. As shown from the statistical graph, Bacteroides fragilis and inactivated Bacteroides fragilis significantly increase the amount of the CD8⁺ T cells in tumor microenvironment. In the statistical analysis graph, * represents student t-test p<0.05, ** represents student t-test p<0.01. p<0.05 represents statistically significant difference. There are 12 mice in each treated group.

FIG. 7 is a typical analysis graph of flow cytometry of one mouse in each group after Akkermansia muciniphila is administrated to mice implanted with breast cancer cells, wherein figures of the right quadrant show the percentages of CD8⁺ T cells in cells in tumor microenvironment. As shown from the quadrant graph in analysis graph of the flow cytometry, Akkermansia muciniphila increases the relative amount of the CD8⁺ T cells by more than 13 times compared with the saline control group. FIG. 8 is a statistical analysis graph of the percentage of CD8⁺ T cells in cells in tumor microenvironment after Akkermansia muciniphila is administrated to mice implanted with breast cancer cells. As shown from the statistical graph, Akkermansia muciniphila significantly increase the amount of the CD8⁺ T cells in tumor microenvironment. In the statistical analysis graph, *** represents student t-test p<0.001. p<0.001 represents statistically significant difference. There are 12 mice in each treated group.

The results show that either Bacteroides fragilis and inactivated Bacteroides fragilis or Akkermansia muciniphila has a significant role in inhibiting the formation and growth of tumor in mice (FIGS. 4, 5, 9 and 10). In addition, the results of FIGS. 4, 5, 9 and 10 show the tumor size in mice treated with Bacteroides fragilis or Akkermansia muciniphila by gavage is significantly smaller than that in saline control group, indicating that Bacteroides fragilis and inactivated Bacteroides fragilis of Akkermansia muciniphila promotes the infiltration and/or accumulation of CD8⁺ cell in tumor microenvironment to enhance anti-tumor effect in the body, and inhibits the tumor growth, having a good effect on the prevention and treatment of tumors, such as breast cancer.

Although the present invention has been disclosed In the form of preferred embodiments and variations thereon, it will be understood that numerous additional modifications and variations could be made thereto without departing from the scope of the invention.

For the sake of clarity, it is to be understood that the use of ‘a’ or ‘an’ throughout, this application does not exclude a plurality, and ‘comprising’ does not exclude other steps or elements.

1. A method of preventing or treating a tumor, comprising administering Bacteroides fragilis or Akkermansia muciniphila to a subject in need thereof.

2. The method according to claim 1, wherein the Bacteroides fragilis or Akkermansia muciniphila is at least one of live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and culture supernatant of Bacteroides fragilis or Akkermansia muciniphila.

3. The method according to claim 1, wherein the tumor is a solid tumor.

4. The method according to claim 1, wherein the tumor is breast cancer, or at least one of a liver, lung, skin, oral, esophagus, stomach, intestine, kidney, prostate, brain, nervous system, bladder, lymph, and pancreas tumor.

5. A pharmaceutical composition for as least one of preventing and treating tumors, comprising a pharmaceutically effective amount of Bacteroides fragilis or Akkermansia muciniphila and a pharmaceutically acceptable carrier thereof, wherein the pharmaceutical composition promotes at least one of infiltration and accumulation of CD8 positive cytotoxic T lymphocytes in tumor microenvironment.

6. The pharmaceutical composition according to claim 5, wherein the Bacteroides fragilis or Akkermansia muciniphila is at least one of live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and culture supernatant of Bacteroides fragilis or Akkermansia muciniphila.

7. A foodstuff for at least one of preventing and treating tumors, including Bacteroides fragilis or Akkermansia muciniphila, wherein the foodstuff promotes at least one of infiltration and accumulation of CD8 positive cytotoxic T lymphocytes in tumor microenvironment.

8. The foodstuff according to claim 7, wherein the Bacteroides fragilis or Akkermansia muciniphila is at least one of live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and culture supernatant of Bacteroides fragilis or Akkermansia muciniphila.

9. A food additive for at least one of preventing and treating tumors, including Bacteroides fragilis or Akkermansia muciniphila, wherein the food additive promotes at least one of in filtration and accumulation of CD8 positive cytotoxic T lymphocytes in tumor microenvironment.

10. The food additive according to claim 9, wherein the Bacteroides fragilis or Akkermansia muciniphila is at least one of live bacterium of Bacteroides fragilis or Akkermansia muciniphila; genetically recombined, altered or modified, attenuated, chemically treated, physically treated, or inactivated Bacteroides fragilis or Akkermansia muciniphila; lysate of Bacteroides fragilis or Akkermansia muciniphila; and culture supernatant of Bacteroides fragilis or Akkermansia muciniphila.

11. The method according to claim 1, wherein the Bacteroides fragilis or Akkermansia muciniphila promotes at least one of infiltration and accumulation of CD8 positive cytotoxic T lymphocytes in tumor microenvironment.
